(19) United States
(12) Patent Application Publication
(10) Pub. No.: US 2025/0069757 A1
(43) Pub. Date: Feb. 27, 2025

(54) METHOD OF PREDICTING ADAS-COG SCORE

(71) Applicant: WisTa Laboratories Ltd., Singapore (SG)

(72) Inventors: Anish Agarwal, Nweton, MA (US); Vishal Misra, New York, NY (US); Bjoern Schelter, Aberdeen, Aberdeenshire (GB); Devavrat Shah, Waban, MA (US); Dennis Shen, Irvine, CA (US); Helen Shiells, Aberdeen, Aberdeenshire (GB); Claude Michel Wischik, Aberdeen, Aberdeenshire (GB)

(73) Assignee: WisTa Laboratories Ltd., Singapore (SG)

(21) Appl. No.: 18/725,043

(22) PCT Filed: Dec. 13, 2022
(86) PCT No.: PCT/EP2022/085684

§ 371 Date: Jun. 27, 2024

(30) Foreign Application Priority Data
Dec. 29, 2021 (GB) 2119104.4
Aug. 3, 2022 (GB) 2211324.5

(51) Int. Cl. G16H 50/50; A61B 5/00; G16H 10/20; G16H 50/70

(52) U.S. Cl. CPC G16H 50/50; A61B 5/4088; G16H 50/70; G16H 10/20

(57) ABSTRACT

A method for predicting a patient or patient cohort's cognition score on the Alzheimer's Disease Assessment Scale-cognitive subscale (ADAS-Cog). The method comprises obtaining data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans; encoding the data into a tensor across patients or patient cohorts, time and treatment plan; generating a synthetic model of a target patient or target patient cohort using a machine learning process and the tensor; and predicting an ADAS-Cog score of the target patient or target patient cohort under a target treatment plan selected from the plurality of treatment plans, using the synthetic model.

### FIELD OF THE INVENTION

The present invention relates to a method for predicting a level of cognitive function and particularly, although not exclusively, to a method for predicting a patient or patient cohort's cognition score on the Alzheimer's Disease Assessment Scale-cognitive subscale (ADAS-Cog).

### BACKGROUND

Alzheimer's disease is a neurological disease whereby excess protein builds up in the brain, impairing neuronal function and eventually leading to cell death. The disease is characterised by continual progression, but the rate of progression is individual.

The Alzheimer's Disease Assessment Scale-cognitive subscale (ADAS-Cog) was developed in the 1980s to track the rate of progression of Alzheimer's disease for an individual, and in particular to determine and track an individual's level of cognitive and non-cognitive function. Since then, it has also been used to help in the diagnosis of Alzheimer's, e.g. in pre-dementia populations. ADAS-Cog is now considered the gold standard for assessing the efficacy of treatments for dementia, including Alzheimer's disease.

The diagnosis of Alzheimer's disease, and the monitoring of a subject's mental capacity once diagnosed and under particular treatments, is an active and multi-faceted area of research, and is often the subject of clinical trials.

Pharmaceutical companies are increasingly using advanced analytics to help run clinical trials.

This trend has been buoyed by a growing interest in personalized healthcare, and the rapid development of modern statistical techniques and computing resources with the advent of machine learning (Shah et al, 2019; Bhatt, 2021). Improving the efficiency of clinical trials by adapting their design and analysis is a particular interest. Furthermore, dropouts (i.e., patients who do not stay for the full course of a clinical trial) are a large source of bias in trials, and can be a significant reason why trials fail (Little et. al, 2012).

The present invention has been devised in light of the above considerations.

### SUMMARY OF THE INVENTION

In a first aspect, there is provided a method for predicting a patient or patient cohort's cognition score on the Alzheimer's Disease Assessment Scale-cognitive subscale (ADAS-Cog), the method comprising:
obtaining data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans;
encoding the data into a tensor across patients or patient cohorts, time and treatment plan;
generating a synthetic model of a target patient or target patient cohort using a machine learning process and the tensor; and
predicting an ADAS-Cog score of the target patient or target patient cohort under a target treatment plan selected from the plurality of treatment plans, using the synthetic model.

The present inventors have found that this data relating to a plurality of patients' ADAS-Cog trajectories when undergoing a plurality of different treatment plans (e.g. from clinical trials for the treatment of Alzheimer's disease) surprisingly displays significant latent linear structure in the induced tensors, despite patients with Alzheimer's disease having heterogenous health trajectories. This is particularly surprising at the individual patient level. This means that there are a limited canonical patient (or patient cohort) profiles such that all patients can be expressed as linear combinations of these profiles with respect to their ADAS-Cog score. Consequently, this enables the prediction of the ADAS-Cog trajectory associated with any patient under a particular treatment plan by leveraging information from other patients that underwent that same treatment plan.

In this way, the ADAS-Cog score, or longitudinal trajectory of the ADAS-Cog score, of the target patient or target patient cohort under an unseen treatment plan (i.e. a treatment plan which the target patient has not undertaken) may be estimated.

It should be understood that the target treatment is generally an unseen treatment plan, i.e. a treatment plan which the target patient has not undertaken, but for which a prediction of what would have been the outcome if the patient had undertaken the target treatment plan, is wanted.

Optional features will now be set out. These are applicable singly or in any combination with any aspect of the present disclosure.

It is to be understood that the synthetic model is a model which uses available data to create synthetic trajectories (e.g. of the ADAS-Cog score over time for patient/patient cohorts and treatment combinations) that are unseen in the available data.

Preferably, the data relating to the plurality of patients or patient cohorts is data from a clinical trial for treating Alzheimer's disease.

It is to be understood that the method may not be limited to a method for predicting ADAS-Cog score, but may be applied to the prediction of other scores for predicting neurocognitive state or decline, such as Mini-Mental State Examination (MMSE), Global Deterioration Scale (GDS) or Montreal Cognitive Assessment (MoCA).

As such, more generally, the method may be a method for predicting a patient or patient cohort's neurocognitive state. The obtained data may thus include information relating to the longitudinal trajectories of the plurality of patients', or patient cohorts' neurocognitive state over time. The target patient or target patient cohort may be undergoing an actual treatment plan, wherein the actual treatment plan is different from the target treatment plan. In particular, the target patient/cohort may be undergoing an “actual” treatment plan, wherein the outcome of that first treatment plan can be observed. The outcome of the target patient/cohort under a second, different “target” treatment plan, which the target patient/patient cohort has not undertaken, can be predicted using the synthetic model. Here, the term “outcome” is to be understood as the ADAS-Cog score of the target patient/cohort at time t, or the ADAS-Cog trajectory of the target patient/cohort over time.

The tensor may be an order-three tensor.

The data in the tensor may include, for each patient of the plurality of patients/cohorts, data relating to (i) the ADAS-Cog trajectory of the respective patient/cohort over time (e.g. a baseline measurement (e.g. at t=0) of ADAS-Cog score of the respective patient, and the ADAS-Cog score of the patient/cohort at a plurality of times/visits thereafter); and (ii) the treatment plan undertaken by the respective patient/cohort.

One or more of the plurality of treatment plans may include one or more different neuropharmacological interventions, such as a pharmaceutical or therapy, e.g. for the treatment of Alzheimer's disease. A treatment plan in the plurality of treatment plans may correspond to no treatment (e.g. such that patient undergoing this treatment plan are in a control group). The different treatment plans may include different dosage regimes of the treatment/pharmaceutical. For example, a first treatment plan may correspond to a high dose of a treatment in combination with use of a standard care of therapy, a second treatment plan may correspond to a high dose of the treatment in combination with no use of the standard care of therapy, a third treatment plan may correspond to a low dose of the treatment in combination with use of the standard care of therapy, and a fourth treatment plan may correspond to a low dose of the treatment in combination with no use of the standard care of therapy.

The synthetic model of the target patient or target patient cohort may be generated using data pertaining to the target patient or target patient cohort. The data pertaining to the target patient or target patient cohort may include data relating to (i) the ADAS-Cog trajectory of the target patient/cohort over time (e.g. a baseline measurement (e.g. at t=0) of ADAS-Cog score of the target patient/cohort, and the ADAS-Cog score of the target patient/cohort at a plurality of times/visits thereafter); and (ii) the actual treatment plan undertaken by the respective patient.

If the data is pertaining to a patient cohort rather than an individual patient, the respective ADAS-Cog scores may be an average (e.g. mean) ADAS-Cog score over the cohort.

Preferably, the synthetic model may be generated using the machine learning process based, at least in part, on a set of observed data in the tensor corresponding to a same treatment plan as the actual treatment plan undertaken by the target patient or target patient cohort. In particular, the synthetic model may be learned, at least in part, from the set of observed data from the tensor pertaining to the actual treatment plan undertaken by the target patient/cohort. This data may include a set of observed pairs (time, treatment plan) of data that the target patient/cohort, and the patient data in the tensor, have in common. In other words, the machine learning process may be based on the set of (trial visit, treatment plan) data pairs for the target patient/cohort, and those patients in the tensor that correspond to a same visit time (e.g. day) and under the same (actual) treatment plan.

The machine learning process may comprise determining a minimum-norm linear relationship between the data pertaining to the target patient/cohort and the set of observed data in the tensor associated with the actual treatment plan.

The machine learning process may comprise performing a principal component regression (PCR) analysis. In particular, the machine learning process may include performing PCR between the data pertaining to the target patient/cohort and the set of observed data in the tensor corresponding to the actual treatment plan undertaken by the target patient/cohort. The PCR process may define a unique minimum-norm linear relationship between the data pertaining to the target patient/cohort and the set of observed data in the tensor associated with the actual treatment plan.

Alternatively, other known machine learning algorithms (parametric or non-parametric) could be used in the machine learning process to learn a relationship between the data pertaining to the target patient/cohort and the set of observed data in the tensor associated with the actual treatment plan. For example, a neural network or a random forest may be used.

Optionally, the method may comprise validating the synthetic model of the target patient/cohort. This may include determining a training error of the learned synthetic model and only proceeding to the step of predicting the ADAS-Cog score of the target patient/cohort using the synthetic model if the determined training error meets a predefined criterion/threshold. In this way, it can be verified that the synthetic model is trained to a satisfactory standard. Furthermore, this step also verifies that the underlying data likely satisfies the desirable properties to provide an accurate estimation.

The step of predicting the ADAS-Cog score of the target patient/cohort under the target treatment plan may be based on the learned synthetic model, and a set of data in the tensor corresponding to the target treatment plan.

The prediction may be a prediction of the ADAS-Cog trajectory of the target patient/cohort under the target treatment plan over time, or may be a prediction of the ADAS-Cog score at a particular time t.

Optionally, the method may include predicting an ADAS-Cog score of the target patient/cohort under each treatment plan of the plurality of treatment plans using the synthetic model.

As mentioned above, the data may be from a clinical trial for treating Alzheimer's disease; e.g. for assessing the efficacy of a neuropharmacological intervention (such as a pharmaceutical), in the treatment of Alzheimer's disease. The plurality of patients may be a treatment group who have been diagnosed with Alzheimer's disease. The plurality of patients may include patients who have not been diagnosed with Alzheimer's disease.

The above method may be computer-implemented. For example, the method may be implemented on one or more computer processing devices. The one or more computer processing devices may be one or more computers, servers, cloud-based devices, for example.

The method may further comprise using the predicted ADAS-Cog score of the target patient or target patient cohort under the target treatment plan in results of a clinical trial.

As a first example, the method may be for predicting missing data in a clinical trial due to a dropout (i.e. a patient who has not stayed for the full course of a clinical trial). As such, the target patient may be a dropout of a clinical trial, and the method may further comprise using the predicted ADAS-Cog score of the target patient as the result for the target patient in the clinical trial results.

In this way, even if a patient has dropped out of the trial such that their full ADAS-Cog trajectory over time, and thus outcome of the trial, cannot be observed, their ADAS-Cog score over time can be predicted by leveraging the observations of the other patients in the trial. Dropouts are generally a large source of bias in trials, and can be a significant reason why trials fail. This method reduces this bias.

The method of the present aspect may include any combination of some, all or none of the above described preferred and optional features.

As another example, the method may be used to design data-efficient clinical trials for personalised treatments at the patient cohort level.

In particular, in a second aspect of the invention there is provided a method for processing data for a clinical trial for treating a neurocognitive disease using the above described method, the method comprising:
obtaining data relating to at least two patient cohorts, wherein a first patient cohort includes a first subset of patients undergoing a first treatment plan and a second subset of patients undergoing a second treatment plan, and a second patient cohort includes a first subset of patients undergoing the first treatment plan and a second subset of patients undergoing a third treatment plan, wherein the data includes information on the longitudinal trajectories of the patient cohorts' ADAS-Cog score over time;
encoding the data into a tensor across patient cohorts, time and treatment plan;
generating a first synthetic model of the first patient cohort and a second synthetic model of the second patient cohort using a machine learning process and the tensor; and
predicting an ADAS-Cog score of the first patient cohort under the third treatment plan using the first synthetic model, and an ADAS-Cog score of the second patient cohort under the second treatment plan using the second synthetic model.

In this way, the first/second synthetic model can predict the ADAS-Cog scores (or ADAS-Cog trajectories over time) for every patient cohort under unseen treatments. This could result in the design of clinical trials including many fewer patients/subjects, as the outcome of subjects could be predicted by leveraging corresponding outcomes from a smaller subset of subjects that have undergone the clinical trial. This would result in a more data efficient clinical trial.

The clinical trial may be a trial for treating Alzheimer's, or behavioural variant fronto-temporal dementia (FTD), for example.

The tensor may be an order-three tensor.

The data in the tensor may include, for each patient in the patient cohorts, data relating to (i) the ADAS-Cog trajectory of the respective patient over time (e.g. a baseline measurement (e.g. at t=0) of ADAS-Cog score of the respective patient, and the ADAS-Cog score of the patient at a plurality of times/visits thereafter); and (ii) the treatment plan undertaken by the respective patient.

The treatment plans may include one or more different neuropharmacological interventions, such as a pharmaceutical or therapy, e.g. for the treatment of Alzheimer's disease. One of the treatment plans may correspond to no treatment (e.g. such that patient undergoing this treatment plan are in a control group). The different treatment plans may include different dosage regimes of the treatment/pharmaceutical. For example, the first treatment plan may correspond to a high dose of a treatment in combination with use of a standard care of therapy, the second treatment plan may correspond to a high dose of the treatment in combination with no use of the standard care of therapy, and the third treatment plan may correspond to a low dose of the treatment in combination with use of the standard care of therapy. A fourth treatment plan may correspond to a low dose of the treatment in combination with no use of the standard care of therapy.

The first/second synthetic model may be generated using data pertaining to the first/second patient cohort, respectively. The data pertaining to the first/second patient cohort may include data relating to (i) the ADAS-Cog trajectory of the first/second patient cohort over time (e.g. a baseline measurement (e.g. at t=0) of ADAS-Cog score of the first/second patient cohort, and the ADAS-Cog score of the first/second patient cohort at a plurality of times/visits thereafter); and (ii) the actual treatment plan undertaken by the patients in the respective patient cohort.

The respective ADAS-Cog scores may be an average (e.g. mean) ADAS-Cog score over the cohort.

Preferably, the first/second synthetic model may be generated using the machine learning process based, at least in part, on a set of observed data in the tensor corresponding to a same treatment plan as the actual treatment plan undertaken by the patients in the first/second patient cohort, respectively. For example, the first synthetic model may be generated using the machine learning process based, at least in part, on a set of observed data in the tensor corresponding to the first treatment plan (as a subset of patients in both the first and second patient cohorts undergo the first treatment plan).

The machine learning process may comprise determining a minimum-norm linear relationship between the data pertaining to the first/second patient cohort and the set of observed data in the tensor associated with the subgroup of patients in the first/second cohort undergoing the first treatment plan.

The machine learning process may comprise performing a principal component regression (PCR) analysis. In particular, the machine learning process may include performing PCR between the data pertaining to the first/second patient cohort and the set of observed data in the tensor corresponding to the first treatment plan undertaken by the subset of patients in the first/second patient cohort respectively. The PCR process may define a unique minimum-norm linear relationship between the data pertaining to the first/second patient cohort and the set of observed data in the tensor associated with the first treatment plan undertaken by the respective subset of patients in the first/second patient cohort, respectively.

Alternatively, other known machine learning algorithms (parametric or non-parametric) could be used in the machine learning process to learn the linear relationship. For example, a neural network or a random forest may be used.

Optionally, the method may comprise validating the first and/or second synthetic model. This may include determining a training error of the learned first/second synthetic model and only proceeding to the step of predicting the ADAS-Cog scores of the target patient/cohort using the synthetic model if the determined training error meets a predefined criterion/threshold. In this way, it can be verified that the synthetic models are trained to a satisfactory standard. Furthermore, this step also verifies that the underlying data likely satisfies the desirable properties to provide an accurate estimation.

The step of predicting the ADAS-Cog score of the first/second patient cohort under the third/second treatment plan may be based on the learned first/second synthetic model, and a set of data in the tensor corresponding to the third/second treatment plan, respectively.

The prediction may be a prediction of the ADAS-Cog trajectory of the first/second patient cohort under the third/second treatment plan over time, or may be a prediction of the ADAS-Cog score at a particular time t.

The above method may be computer-implemented. For example, the method may be implemented on one or more computer processing devices. The one or more computer processing devices may be one or more computers, servers, cloud-based devices, for example.

The method of the present aspect may include any combination of some, all or none of the above described preferred and optional features.

In a third aspect, there is provided a system including one or more processors and a memory, the memory containing machine executable instructions which, when executed on the one or more processors, cause the one or more processors to perform the method of the first aspect.

The memory may contain machine executable instructions which, when executed on the processor, cause the processor to perform the method of the first aspect including any one, or combination insofar as they are compatible, of the optional features set out with reference thereto.

As such, there is provided a system for predicting a patient or patient cohort's cognition score on the Alzheimer's Disease Assessment Scale-cognitive subscale (ADAS-Cog), the system comprising:
data acquisition means, configured to obtain data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans;
tensor generating means, configured to encode the data into a tensor across patients or patient cohorts, time and treatment plan;
synthetic model generating means, configured to generate a synthetic model of a patient or patient cohort using a machine learning process and the tensor; and
predicting means configured to predict an ADAS-Cog score of the patient or patient cohort under a target treatment plan of the plurality of treatment plans using the synthetic model.

In some embodiments, the system may comprise one or more computers, servers, or cloud-based devices, for example.

They system may be a system for processing data for a clinical trial for treating Alzheimer's disease, wherein:
the data acquisition means are configured to collect data from at least two patient cohorts, wherein a first patient cohort includes a first subset of patients undergoing a first treatment plan and a second subset of patients undergoing a second treatment plan, and a second patient cohort includes a first subset of patients undergoing the first treatment plan and a second subset of patients undergoing a third treatment plan, wherein the data includes information on the longitudinal trajectories of the patient cohorts' ADAS-Cog score over time;
the tensor generating means are configured to encode the data into a tensor across patient cohorts, time and treatment plan;
the synthetic model generating means are configured to generate a first synthetic model of the first patient cohort and a second synthetic model of the second patient cohort using a machine learning process and the tensor; and
the predicting means are configured to predict an ADAS-Cog score of the first patient cohort under the third treatment plan using the first synthetic model, and an ADAS-Cog score of the second patient cohort under the second treatment plan using the second synthetic model.

Accordingly, the system may be configured to perform the method of the second aspect including any one, or combination insofar as they are compatible, of the optional features set out with reference thereto.

According to a fourth aspect, there is provided a non-transitory computer readable storage medium containing machine executable instructions which, when executed on a processor, cause the processor to perform the method of the first aspect or the second aspect, including any one, or any combination insofar as they are compatible, of the optional features set out with reference thereto.

According to a fifth aspect, there is provided a computer program comprising executable code which, when run on a computer, causes the computer to perform the method of the first aspect or the second aspect.

According to a sixth aspect, there is provided a computer readable storage medium storing a computer program comprising code which, when run on a computer, causes the computer to perform the method of the first aspect or the second aspect.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 includes two graphs showing the spectra of order-3 tensors of clinical trial data (i) at the patient cohort level under mode-2 unfolding; and (ii) at the individual patient level under “high-using”. respectively;

FIG. 2 includes two tables showing actual and data-efficient trial data, respectively;

FIG. 3 is a graph showing the errors associated with a data-efficient trial;

FIG. 4 includes plots showing the average mean absolute errors and standard errors of imputing missing data for dropouts of a clinical trial across different treatments and varying numbers of observed treatments using a method according to an aspect of the disclosure compared to other baseline measurement methods;

FIG. 5 includes plots showing a comparison between the observed density associated with compliers of a clinical trial, with the estimated density associated with dropouts as predicted using a method according to an aspect of the disclosure;

FIG. 6 is a flow diagram of a method for predicting a patient or patient cohort's cognition score on the Alzheimer's Disease Assessment Scale-cognitive subscale (ADAS-Cog); and

FIG. 7 is a schematic of a system for performing the method of FIG. 6.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The inventors carried out an analysis of the above method on real clinical trial data to ascertain that clinical trial data can be used with a synthetic interventions (SI) estimator designed to recover low-rank tensors with missing not at random data (i.e., is causally valid).

### Clinical Trial Overview

Data from a phase III clinical trials (TRx-237-005 and TRx-237-015) for Alzheimer's disease was obtained. These clinical trials involved 1162 patients. Before the trials began, each patient's baseline measurements were collected, which included the patient's sex, age, baseline ADAS-Cog and mini-mental state examination (MMSE) score.

Hereinafter, Xᵢ is denoted as the vector of baseline measurements associated with patient i.

Across the two phase III studies, each patient was randomly assigned to a treatment of 8, 150, 200 or 250 mg/day of LMTM (developed by TauRx Therapeutics), but a lack of dose-response was observed in both trials. Further population pharmacokinetic analyses showed steep concentration-response relationships for steady-state plasma levels in the 8 mg/day range. Patients were subsequently categorised into those with ‘low’ and ‘high’ plasma levels using a threshold based on the lower limit of quantitation of the assay on Day 1. This analysis yielded highly significant differences in cognitive decline and brain atrophy in the group with ‘high’ plasma levels (Schelter et. al., 2019). Additionally, each patient entered the trial either already using the standard care of therapy or not. This yielded D=4 possible combinations: “low-using”, “low-none”, “high-using” and “high-none”. The trial spanned 5 visits with 13 weeks in between each visit. At each visit t, every patient i's progressive ADAS-Cog score, Yₜᵢ∈ [symbol] , was measured and recorded. For completeness, it is noted that a positive change in ADAS-Cog score from baseline indicates that the disease is worsening.

Encoding Clinical trial data as a tensor

The clinical trial data can be encoded both at a patient cohort and on an individual patient level, as an order-3 tensor.

To demonstrate this, the clinical trial data was first encoded in an order-3 tensor at the patient cohort level. Before the trial began, the patients were clustered into 6 cohorts based on the baseline measurements Xᵢ. The goal was to estimate the causal effect of all four treatment combinations on each of the 6 patient cohorts. The clusters were not formed using observed trial visit outcomes (i.e., Yₜᵢ). For each patient cohort, the ADAS-Cog trajectories were observed under every treatment combination (with the exception of one cluster in which none of the patients underwent the treatment “low-none”). This data was encoded into an order-three tensor, Y₁∈ [symbol] ^{6×5×4}, where [Y₁]_{ijd} refers to the observed mean ADAS-Cog trajectory for patient cohort i at visit j under treatment d.

Next, the data on an individual patient level was analysed, rather than at a cohort level. The available individual patient ADAS-Cog trajectory data was encoded into an order-three tensor, Y₂∈ [symbol] ^{1162×5×4}, where [Y₂]_{ijd} refers to the patient i at visit j under treatment d. Unlike Y₁, Y₂ is only partially observed since each patient can only undergo a single treatment. However, for every treatment d, if only those patients within treatment arm d are considered, [symbol] _{d}, then the resulting matrix, denoted by Y₂^{d}∈ [symbol] ^{N}^{×5} is fully observed; here N_{d}=| [symbol] _{d}|. [symbol] _{d} only included patients that attended all five visits.

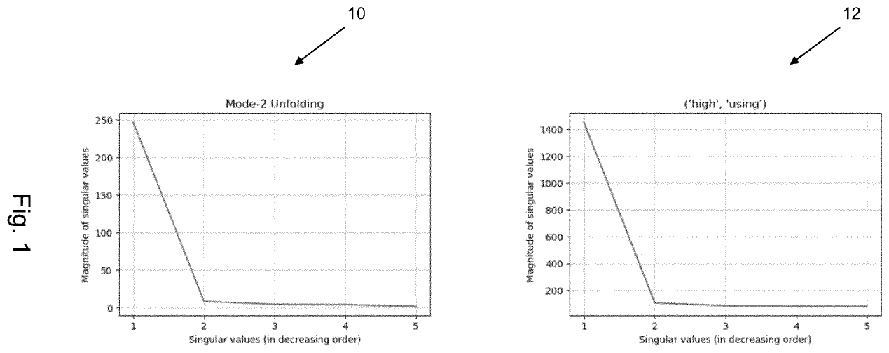

The spectral profile of Y₁ was analysed by inspecting its mode-1 and mode-2 unfoldings, as shown in plot 10 of FIG. 1. Further details of tensor unfoldings can be found at Section 7 of Agarwal, A et al., (2020). As shown in plot 10, it was found that Y₁ in effect has a low canonical polyadic (CP) tensor rank of 1. Y₂ was also analysed. Plot 12 of FIG. 1 shows the spectral profile of Y₂^{d} under “high-using” treatment. It was shown that, as with Y₁, Y₂^{d} is also approximately rank 1.

As both Y₁ and Y₂^{d} are both low-rank tensors, it was found that there is significant latent linear structure in the induced tensors/matrices. Specifically, it means that there are only a limited number of canonical patient, or patient cohort, profiles such that all patients can be expressed as linear combinations of these profiles with respect to their ADAS-Cog score. This is surprising given the heterogenous health trajectories of patients with Alzheimer's disease. Consequently, the present inventions found that this enables the prediction of ADAS-Cog trajectory associated with any patient under a particular treatment by leveraging information from other patients that underwent that treatment using known synthetic intervention estimators.

### Trajectory Prediction via Synthetic Interventions

Without loss of generality, the aim of the above method is to predict the ADAS-Cog score for patient (or cohort) i on visit t under treatment d, which we denote as Yₜᵢ^{(d)}.

A two-step Synthetic Intervention Estimator is used to estimate the heterogeneous ADAS-Cog trajectory of each patient under each treatment. An example of such a two-step SI estimator is disclosed in Agarwal, A et al., (2020).

A first step builds a “synthetic” model of the target patient i, parametrized by a machine learning model, ŵ^{(i,d)}, which is trained based on the common observations between patient i and the patients in [symbol] _{d}. A second step leverages observed outcomes of patients in [symbol] _{d} and the learned synthetic model to simulate patient i′s ADAS-Cog score under treatment d at time t.

The notation used to aid understanding is set out below.

Let Ω_{d}⊂[T]×[D] denote the set of observed (trial visit, treatment) pairs that patient i and those patients in [symbol] _{d} have in common, i.e., the set of (trial visit, treatment) pairs for patient i and those in [symbol] _{d} that are for the same visit day and under the same treatment; let T_{d}=|Ω_{d}|.

Let Yᵢ=[Y_{τi}^{(a)}: (τ, α)∈Ω_{d}]∈ [symbol] ^{K} and Zᵢ=[Yᵢ, Xᵢ]∈ [symbol] ^{T}^{_{d}}^{+K} denote the concatenation of baseline measurements Xᵢ∈ [symbol] ^{K} with Yᵢ.

Similarly, for those patients within [symbol] _{d}, define Y [symbol] _{_{d}}=[Y_{τj}^{(α)}(τ, α)∈Ω_{d}, j∈ [symbol] _{d}]∈ [symbol] ^{T}^{_{d}}^{+N}^{_{d}}, X [symbol] _{_{d}}=[Xⱼ:j∈ [symbol] _{d}]∈ [symbol] ^{K}, and Z [symbol] _{_{d}}=[Y [symbol] _{_{d}}, X [symbol] _{_{d}}]∈ [symbol] ^{(T}^{_{d}}^{+K)×N}^{_{d}}.

The singular value decomposition is represented as Z [symbol] _{_{d}}=Σ_{l≥z}sₗuₗ⊗vₗ, where ⊗ is the outer product.

The two step SI estimator has a single hyper-parameter k that quantifies the number of singular components of Z [symbol] _{_{d}} to retain. Section 3 of Agarwal, A et al., (2020) provides examples of how k can be chosen.

As mentioned above the first step of the SI estimator, is to build a “synthetic” model of the target patient i, parametrized by a machine learning model, ŵ^{(i,d)}, wherein:

$${\hat{w}}^{( i , d )} = ( {\sum }_{l = 1}^{k} ( \frac{1}{{s}_{l}} ) {v}_{l} ⊗ {u}_{l} ) {Z}_{i}$$

The second step of the SI estimator is to leverage observed outcomes of patients in [symbol] _{d} and the learned synthetic model to simulate patient i's ADAS-Cog score under treatment d at time t:

$${\hat{Y}}_{ti}^{( d )} = {\sum }_{j ∈ {𝒥}_{d}} {\hat{w}}_{J}^{( i , d )} {Y}_{tj}^{( d )}$$

### Data Efficient Clinical Trials for Personalised Treatments

As mentioned, the above described method may be used to design data-efficient clinical trials for personalized treatments at the patient cohort level.

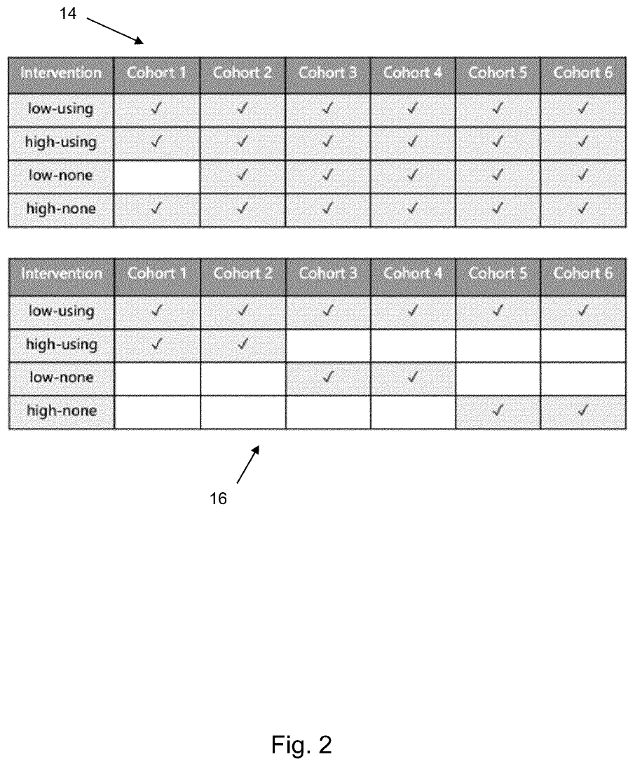

To demonstrate this, the present inventors defined the “low-using” drug combination as the control, since patients in this cohort are essentially on the current standard of care. To simulate a data-efficient trial, the SI estimator described above was allowed to observe ADAS-Cog scores under control for all 6 patient cohorts. However, the SI estimator was only allowed to observe each patient cohort under one of the three other possible treatments (high-using, low-none, high-none). That is, the inventors purposively held out data under these three treatments as a test set to see if the SI estimator can recreate these trajectories. A graphical depiction of the observation patterns for the actual trial data can be seen in table 14 of FIG. 2, and for the simulated data-efficient trial data in table 16 of FIG. 2. In FIG. 2, a tick in a cell represents observed outcomes and bank cells represent unobserved (i.e., held out) outcomes.

Given the sparsity in the pattern in table 16 of FIG. 2, the objective was to estimate the mean ADAS-Cog trajectory for every patient cohort under each of the two treatments that are unobserved, i.e., for an unobserved treatment d, the aim is to estimate:

$${θ}_{i}^{( d )} := \frac{1}{\vert {S}_{i} \vert} {\sum }_{j ∈ {S}_{i}} ( \frac{1}{T} {\sum }_{t \leq T} {Y}_{tj}^{( d )} ) ,$$

where Sᵢ refers to individuals in cohort i.

To quantify the prediction accuracy of the SI estimator for any cohort i and treatment d, a randomized control trial (RCT) estimator was used as the baseline. The RCT estimator takes the average ADAS-Cog outcome across all patients that received treatment d as its prediction. Thus, to predict the ADAS-Cog outcomes of cohort 3 in FIG. 2 under high-using for instance, the RCT estimator will take the average ADAS-Cog outcome of all patients in cohorts 1 and 2. If the patients within the trial were homogeneous, then the RCT estimator would be a strong predictor. More formally, for any cohort i and treatment d, the square error is defined as:

$$S {E}_{i}^{( d )} := 1 - \frac{{( {θ}_{i}^{( d )} - {\hat{θ}}_{i}^{( d )} )}^{2}}{{( {θ}_{i}^{( d )} - ( \frac{1}{{N}_{d}} ) {\sum }_{j ∈ {𝒥}_{d}} {Y}_{t j}^{( d )} )}^{2}}$$

where \hat{θ}ᵢ^{(d)} is the output of the SI estimator. The numerator and denominator above represent the SI and RCT error, respectively. SEᵢ^{(d)} can be interpreted as a modified R² statistic, which captures the gain in accuracy by “personalizing” predictions on a cohort-by-cohort basis.

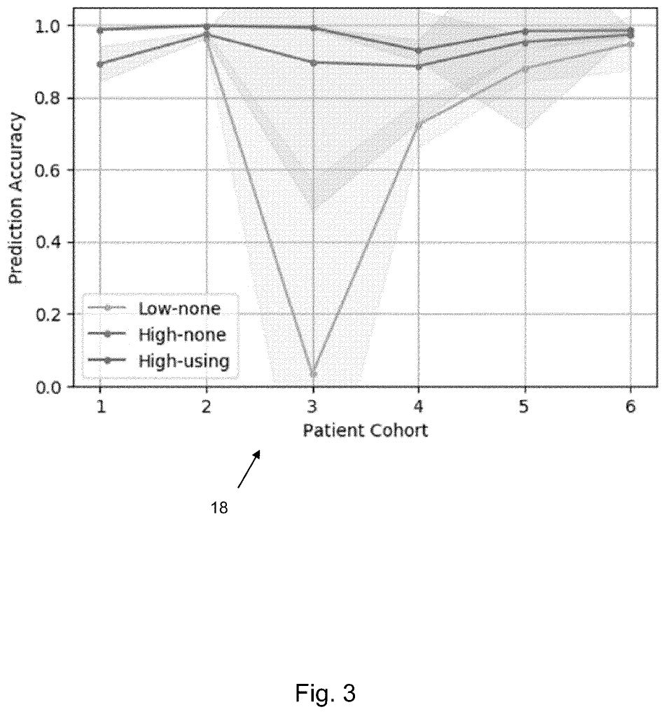

Under the set-up above, the SI estimator was applied to predict the ADAS-Cog trajectories for every patient cohort under unseen treatments. To mitigate randomness, this experiment was repeated 10 times with a different treatment assignment in every iteration. The results can be seen in graph 18 of FIG. 3.

Graph 18 of FIG. 3 shows the square error, SEᵢ^{(d)}, and standard errors (shaded) across each cohort under every treatment.

Across nearly all cohorts i and treatments d, it is found that the median SEᵢ^{(d)}˜0.95. This indicates that the SI estimator far outperforms the RCT estimator. Even in cohort 3 under low-none, where the difference between the RCT estimator and SI estimator is less pronounced, the SI estimator still outperforms the RCT estimator.

This confirms that there is indeed significant heterogeneity between cohorts and remarkably, the SI estimator accurately predicts outcomes despite this heterogeneity and access to only a small subset of data.

### Imputing Missing Values of Dropouts in Clinical Trials

Dropouts (i.e. patients who do not stay for the full course of a clinical trial) are a large source of bias in trials, and can be a significant reason why trials fail (Little et. al., 2012). The present inventors have found that the above method for predicting ADAS-Cog score can be used to impute missing data values due to dropouts

To validate the above described SI estimator, dropouts for patient data that were observed were simulated. To make the simulation realistic, the probably of a patient being a dropout was made a function of their change in ADAS-Cog score. This closely matches reality where patients that tend not to respond to a treatment are more likely to drop out.

The simulation was set up as follows.

T₀<T was chosen as the number of visits at which point no patient has dropped out. Patients were clustered based on their average change in ADAS-Cog score from baseline after visit T₀ into poor, moderate and good responders. Poor responders are patients whose average change from baseline is more than one standard deviation above the average across all patients; good responders are one standard deviation below the average; moderate responders are the remaining patients, i.e., those within one standard deviation. As mentioned above, an increase in ADAS-Cog score means the disease is worsening.

The probability of dropping out after visit T₀ was selected for poor, moderate, and good responders as 60%, 30% and 10% respectively. Due to the small number of patients under the low-none combination, the other three combinations were focussed on.

The aim was to estimate the average change from baseline for each treatment across all patients (simulated compliers and dropouts);

$${μ}^{( d )} := \frac{1}{{N}_{d}} {\sum }_{i ∈ {𝒥}_{d}} ( \frac{1}{T} {\sum }_{t \leq T} {Y}_{ti}^{( d )} - {B}_{i} )$$

where Bᵢ is the measured baseline ADAS-Cog score for patient i before the trial began.

The accuracy was measured via the relative mean absolute error:

$$M A {E}^{( d )} := \frac{\vert {\hat{μ}}^{( d )} - {μ}^{d} \vert}{\vert {μ}^{d} \vert} , {\hat{μ}}^{( d )} = \frac{1}{{N}_{d}} {\sum }_{i ∈ {𝒥}_{d}} ( \frac{1}{T} {\sum }_{t \leq T} ( {\hat{Y}}_{ti}^{( d )} - {B}_{i} ) )$$

Ŷₜᵢ^{(d)} is the output of the SI estimator described above if i is selected as a dropout, and is equal to the observed Yₜᵢ^{(d)} if i is a complier.

The estimates were compared against baseline estimators used in Little et. al., 2012; (i) complete-case: estimates \hat{μ}^{(d)} only using data from compliers; (ii) RCT; (iii) Last Observation Carried Forward (LOCF): imputes missing data for dropouts using the last available outcome (commonly using in longitudinal studies); (iv) multivariate singular spectral analysis (mSSA): imputes missing data for dropouts using a spectral-based time series algorithm (this estimator has proven to outperform many state-of the art time series algorithms on standard benchmarks (Agarwal, et. al., 2021).

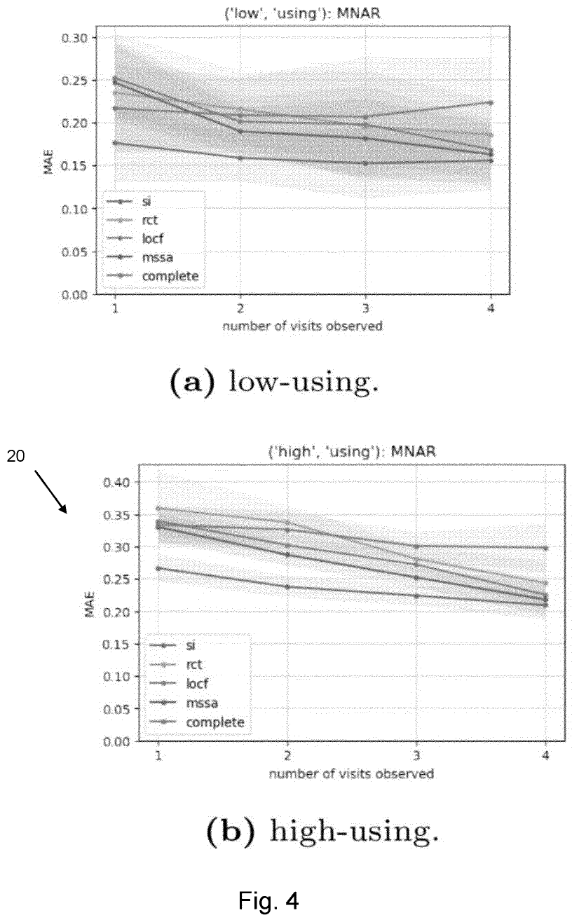

T₀ was varied from 1 to 4. The procedure was repeated 10 times for each T₀, and the resulting average mean absolute errors and standard errors are shown in plot 20 shown in FIG. 4. The standard errors are shown in plot 20 by shaded bands. It was found that the SI estimator not only achieves low relative error, but also consistently outperforms the existing baselines.

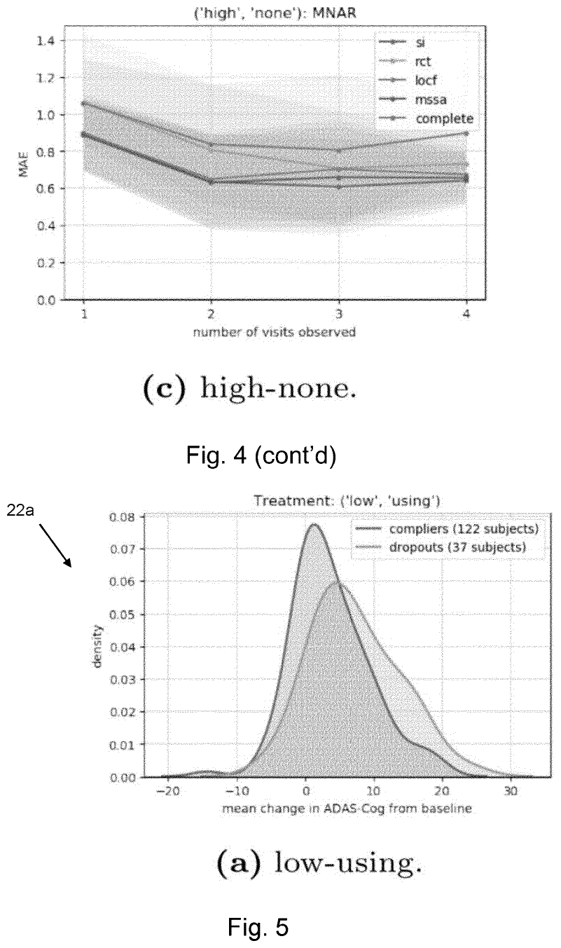

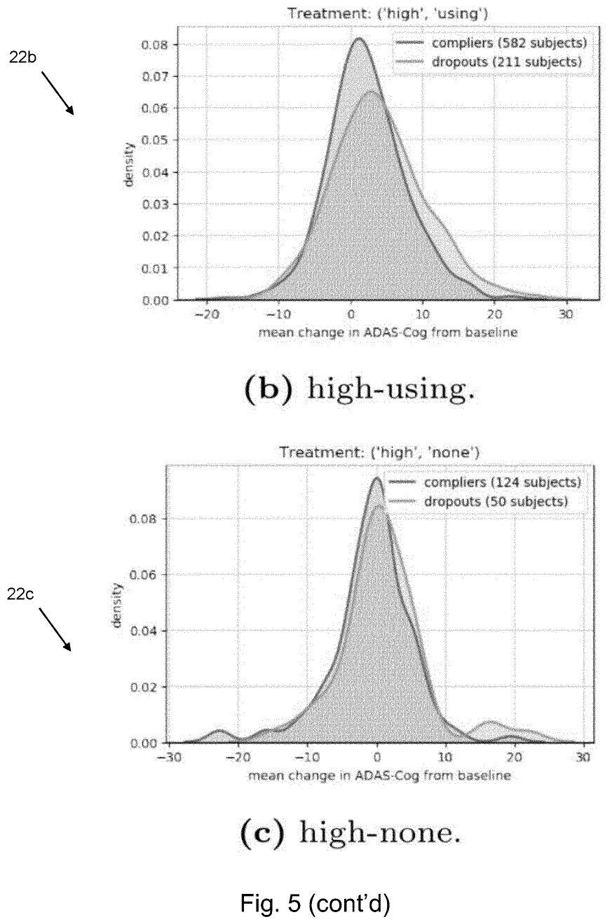

The SI estimator method described above was also used to quantify the bias present due to actual dropouts in the actual clinical trial that was performed by TauRx described above, by building a counterfactual ADAS-Cog trajectory for every dropout patient had they stayed on. In FIG. 5, the distribution of the average change from baseline for compliers (actually observed), and for dropouts (this is a counterfactual distribution created from the SI estimator) is shown. Although the estimated densities cannot be validated as the dropout data is actually missing from the real life trial data, the qualitative conclusions agreed with feedback received from the dropout patients. In particular, plot 22a suggests a natural narrative where the dropouts likely withdrew from the study because they responded poorly to the low-using combination. Indeed, these were patients that vocalized how their assigned combination made them feel unwell. A similar narrative is shown in plot 22b, though the discrepancy between compliers and dropouts is less pronounced. In comparison, plot 22csuggests that the dropouts and compliers responded similarly to the high-none combination. Indeed, many of the dropouts for this particular combination stated that they left the study due to external circumstances such as moving to an inconveniently far location for a new job.

Therefore, the present inventors have shown that the above-described method may be used to accurately impute missing data values due to dropouts.

### Method for Predicting ADAS-Cog Score

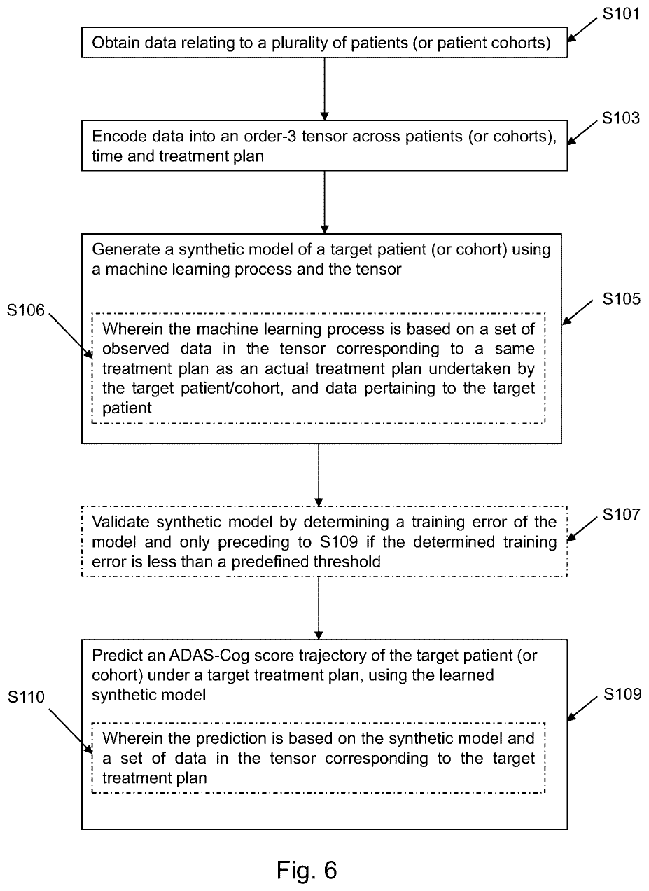

FIG. 6 shows a flow diagram of a method for predicting the ADAS-Cog score of a target patient under an unseen treatment plan. The method may be performed at one or more computing devices.

At S101, data from a clinical trial for treating Alzheimer's disease is obtained (e.g. from an internal or external storage system). The data includes information relating to the longitudinal trajectories of a plurality of patients' ADAS-Cog score over time, and the treatment plan each patient has undergone (e.g. the data includes ADAS-Cog score information, time information, and treatment plan information).

At S103, this data is encoded into an order-3 tensor across patients, time and treatment plan.

At S105, a synthetic model is generated in a first step of a Synthetic Intervention Estimator. The synthetic model is of the target patient, and is generated using a machine learning process. As shown in S106, optionally, the synthetic model is trained based on a set of observed data in the tensor corresponding to the actual treatment plan undertaken by the target patient, and data pertaining to the target patient. For example, the machine learning process may include determining a minimum-norm linear relationship between the data pertaining to the target patient and the set of observed data in the tensor associated with the actual treatment plan undertaken by the target patient. In particular a PCR process may define a unique minimum-norm linear relationship between the data pertaining to the target patient and the set of observed data in the tensor associated with the actual treatment plan.

S107 is an optional step. At S107, the synthetic model is validated to check its accuracy. A training error of the learned synthetic model is computed based on the data pertaining to the target patient, and the synthetic model to test whether the synthetic model can accurately predict the observed data pertaining to the target patient. If the computed error is less than a predefined threshold, the method proceeds to

S109. Otherwise, the method ends.

At S109, an ADAS-Cog trajectory of the target patient under an unseen treatment plan is predicted using the synthetic model, and, as shown in S110, a set of data in the tensor corresponding to the target treatment plan.

In this way, the ADAS-Cog trajectory of a target patient under a particular treatment plan which has not been completed by the target patient can be predicted. This prediction can be used as the outcome of a treatment plan by the target patient in clinical trial results, e.g. if the target patient has dropped out of the clinical trial. This prediction could also be used to provide more data efficient clinical trials (e.g. by predicting some of the results, less subjects in the clinical trial are necessary). This prediction could also be used to design more data efficient clinical trials, or to direct personalised therapy, e.g., by predicting which treatment plans may be more efficient.

Based on the predictions, subpopulation-treatment pairs that yield promising outcomes can be further investigated and pairs that yield undesirable outcomes can be abandoned. In this way, the above method can be used as a recommendation engine to strategically decide the experiments to prioritize and as a tool towards the development of precision medicine.

As one or more of the predictions can be used to forecast an outcome of a clinical trial, the above described method can also be used in early termination applications, e.g. to decide whether to continue or abandon the clinical trial, based on the predicted results.

Further applications of the above method are described below.

A first application is in regard to uncontrolled, single-arm trials, which may be common in rare and/or orphan disease areas. These disease areas have small patient populations and may lack established standard-of-care treatments. These clinical trials may have insufficient statistical power and may raise ethical concerns on administering placebo to patients. In order to address these issues, external control data may be used. This external control data may be used as the data relating to a plurality of patients or patient cohorts in the above described method. In this way, the synthetic model can be used to construct a synthetic twin of every in-trial patient under control using a weighted combination of data from external control patients. The cohort of synthetic twins may then form a synthetic control arm.

Further applications focus on demonstrating real-world safety and effectiveness of an approved treatment. In particular, to provide evidence of safety, a comprehensive surveillance of newly-approved treatments may be performed, to identify potential hazards that could be harmful to patients. This may be known as adverse event reporting, and may be used to determine whether adverse events are caused by a newly-approved treatment. Generally, assessments involve observational studies where the incidence of adverse events is compared between a patient population under a treatment plan, and a natural control group. The above-described method may be used to construct an artificial control group. In particular, for every patient under the treatment, a synthetic twin under control may be constructed from external (e.g. real-world) control data. If the incidence of an adverse event is statistically higher in the observed treatment group compared to the synthetic control group, then it may be determined that the treatment contributes to the adverse event. To demonstrate effectiveness, evidence may be provided that a newly-released treatment is competitive with an established standard-of-care treatment plan. The above-described methods may be used to construct synthetic cohorts under the standard-of-care treatment plan. In particular, for every patient under a new treatment plan, the above described-method may generate a synthetic twin under the standard-of-care treatment plan. The therapeutic outcomes of the two treatment groups may be compared, in order to assess the comparative effectiveness of the new treatment compared to the standard-of-care treatment,

The above-described methods may also be used for label-expansion. For example, in the context of off-label patients (e.g. individuals that are underrepresented or not represented in a randomized control trial), the above-described method may be used to construct, for every off-label patient, a synthetic twin under a (newly released) treatment plan as a weighted combination of data from on-label patients. This may effectively simulate a synthetic trial of off-label patients under the (newly released) treatment plan. This may provide researchers with evidence in support of or against expanding the treatment to the off-label sub-population.

Provided below is an example generalised algorithmic overview of the method of FIG. 6 for constructing synthetic data for a given target “unit” i had it received intervention d, where we assume data associated with unit i is missing. Here, a “unit” may refer to a patient or a patient cohort, and an “intervention” may refer to a treatment plan.

### 1. Data Preparation

a) Prepare a database where for each unit, there is data associated with all interventions that unit goes through.
b) Query database for all observations associated with target unit i.
c) Query database for all observation associated with “donor” units that underwent intervention d.
d) Filter the data so as to identify the collection of observations associated with common interventions between target unit i and the relevant donor units that maximized the minimum between (i) the number of satisfactory observations and (ii) number of filtered donor units.
e) Create a new table that only consists of data pertaining to the common interventions associated with the target unit and filtered donor units (from step (d)); denote these two data structures as y₁ and X₁, respectively.

f) Create a new table that only consists of data pertaining to the interventions associated with the filtered donor units under intervention d; denote this data structure as X₂.

### 2. Data Validation Via Diagnostic Tests

a) Create a new table than concatenates X₁, X₂, i.e. X = [X₁, X2] in a column-wise manner.
b) Perform the singular value decomposition of X and inspect its spectral profile. Check to see if X exhibits low-dimensional structure. If X does not exhibit low-dimensional structure, then X may need to be pre-processed (e.g., apply an autoencoder to X to identify a new low-dimensional representation of X.
c) Perform the subspace inclusion hypothesis test on X₁, X₂, e.g. as detailed in Section 5 of Agarwal et. al., 2020. If the hypothesis test passes, then there is strong diagnostic evidence that we can accurately create synthetic data for unit i under intervention d.

Note, it is set out above in relation to FIG. 1 that data from a clinical trial for treating Alzheimer's disease generally has a low tensor rank, and there is strong diagnostic evidence that we can therefore accurately create synthetic data for unit i under intervention d.

### 3. Synthetic DATA GENERATION

a) Choose a pre-defined training error tolerance ϵ.
b) Follow the synthetic data generation procedure set out above, and further detailed in Section 3 of Agarwal et. al., 2020:
   (i). Model Learning: perform principal component regression (PCR) between y₁ and X₁ to yield \hat{β}ᵢ, which defined the unique minimum-norm linear relationship between the target unit i and filtered donor units. Alternatively, a different machine learning (ML) algorithm (parametric or nonparametric) can be used to learn a relationship between y₁ and X₁ (e.g. using a neural network or a random forest). Denote the learned model between y₁ and X₁ as \hat{f}.
   (ii). Intermediary Synthetic Data Model Validation: compute the training error between the observations y₁ and estimates X₁\hat{β}; if the error is below ϵ, then proceed to the next step as the learnt model between the target unit and donor units is satisfactory, with respect to prediction.
   (iii). Synthetic Data Generation: create synthetic data associated with target unit i under intervention d as X₂\hat{β}. From here, further procedures may be applied on top of estimates X₂\hat{β} (or more generally \hat{f}(X₂)), e,g, by the computing the mean, which expresses the average counterfactual outcome of target i under intervention d.

### 4. Diagnosis of Accuracy of Synthetic Data

a) As an added layer of evaluation, “cross-validation” studies can be performed to investigate whether the steps above are successful in creating the observed dataset. Each donor unit may be iteratively assigned to be the target unit, and the remaining donor units then form the donor groups for that particular intervention. That is, the temporary target unit's observations under intervention d can be observed (i.e., have access to the “synthetic” data which is to be reproduced). The same procedure described above can then be carries out with the extra validation of measuring the prediction error between X₂\hat{β} (or more generally \hat{f}(X₂)), where X₂ is now defined over the temporary donor group, and the observations associated with the temporary target unit under intervention d.

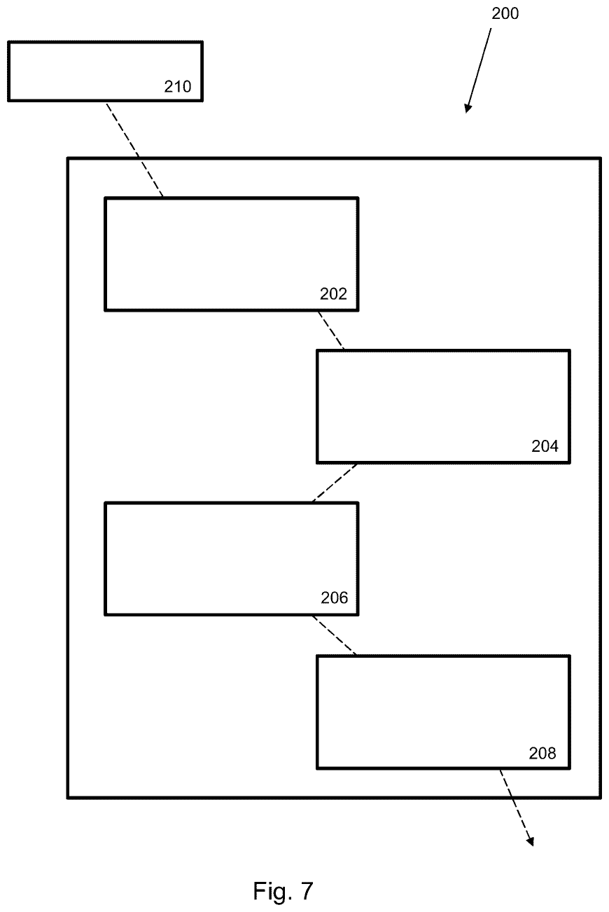

FIG. 7 is a schematic of a system 200 for performing the method of FIG. 6. The system 200 may comprise one or more computing devices, for example.

The system 200 comprises data acquisition means 202, configured to obtain data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans. The data may be obtained from an external storage medium 210 via a wired or wireless connection.

The system 200 also comprises tensor generating means 204, configured to encode the data acquired by the data acquisition means 202 into a tensor across patients or patient cohorts, time and treatment plan.

A synthetic model generating means 206 is configured to generate a synthetic model of a patient or patient cohort using a machine learning process and the tensor generated by the tensor generating means 204. Predicting means 208 is configured to predict an ADAS-Cog score of the patient or patient cohort under a target treatment plan of the plurality of treatment plans using the synthetic model generated by the synthetic model generating means 208. This predicted ADAS-Cog score may be output from the system, e.g. by a wired or wireless connection.

The systems and methods of the above embodiments may be implemented in a computer system (in particular in computer hardware or in computer software) in addition to the structural components and user interactions described.

The term “computer system” includes the hardware, software and data storage devices for embodying a system or carrying out a method according to the above described embodiments. For example, a computer system may comprise a central processing unit (CPU), input means, output means and data storage. Preferably the computer system has a monitor to provide a visual output display. The data storage may comprise RAM, disk drives or other computer readable media. The computer system may include a plurality of computing devices connected by a network and able to communicate with each other over that network.

The methods of the above embodiments may be provided as computer programs or as computer program products or computer readable media carrying a computer program which is arranged, when run on a computer, to perform the method(s) described above.

The term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the invention and the state of the art to which the invention pertains. Full citations for these references are provided below.
Agarwal, A., Shah, D., Shen, D.: Synthetic interventions (2020), arXiv: 2006.07691 [econ.EM]
Bhatt, A.: Artificial intelligence in managing clinical trial design and conduct: Man and machine still on the learning curve? Perspectives in Clinical Research 12(1), 1 (2021)
Little, R. J., D'Agostino, R., Cohen, M. L., Dickersin, K., Emerson, S. S., Farrar, J. T., Frangakis, C., Hogan, J. W., Molenberghs, G., Murphy, S. A., Neaton, J. D., Rotnitzky, A., Scharfstein, D., Shih, W. J., Siegel, J. P., Stern, H.: The prevention and treatment of missing data in clinical trials. New England Journal of Medicine 367(14), 1355-1360 (2012). https://doi.org/10.1056/NEJMsr1203730, pMID: 23034025
Schelter B O, Shiells H, Baddeley T C, Rubino C M, Ganesan H, Hammel J, Vuksanovic V, Staff R T, Murray A D, Bracoud L, Riedel G, Gauthier S, Jia J, Bentham P, Kook K, Storey J M D, Harrington C R, Wischik C M. Concentration-Dependent Activity of Hydromethylthionine on Cognitive Decline and Brain Atrophy in Mild to Moderate Alzheimer's Disease. J Alzheimers Dis. 2019;72(3): 931-946. doi: 10.3233/JAD-190772. PMID: 31658058; PMCID: PMC6918900. (2019)
Shah, P., Kendall, F., Khozin, S., Goosen, R., Hu, J., Laramie, J., Ringel, M., Schork, N.: Artificial intelligence and machine learning in clinical development: a translational perspective. NPJ digital medicine 2(1), 1-5 (2019)

1. A method for predicting a patient or patient cohort's cognition score on the Alzheimer's Disease Assessment Scale-cognitive subscale (ADAS-Cog), the method comprising:
obtaining data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans;
encoding the data into a tensor across patients or patient cohorts, time and treatment plan;
generating a synthetic model of a target patient or target patient cohort using a machine learning process and the tensor; and
predicting an ADAS-Cog score of the target patient or target patient cohort under a target treatment plan selected from the plurality of treatment plans, using the synthetic model.

2. The method of claim 1, wherein the synthetic model of the target patient/cohort is generated using data pertaining to the target patient or target patient cohort.

3. The method of claim 1 or claim 2, wherein the tensor is an order-three tensor.

4. The method of any preceding claim, wherein the target patient or target patient cohort are undergoing an actual treatment plan, wherein the actual treatment plan is different from the target treatment plan.

5. The method of claim 4, wherein the synthetic model is generated using the machine learning process based, at least in part, on a set of observed data in the tensor corresponding to a same treatment plan as the actual treatment plan undertaken by the target patient/cohort.

6. The method of claim 5, wherein the machine learning process comprises determining a minimum-norm linear relationship between the data pertaining to the target patient/cohort and the set of observed data in the tensor associated with the actual treatment plan.

7. The method of any preceding claim, wherein the machine learning process comprises performing a principal component regression, PCR, analysis.

8. The method of any preceding claim, further comprising:
validating the synthetic model of the target patient or target patient cohort, by determining a training error of the generated synthetic model and only preceding to the step of predicting the ADAS-Cog score of the target patient or target patient cohort using the synthetic model if the determined training error meets a predefined criterion.

9. The method of any preceding claim, wherein predicting the ADAS-Cog score of the target patient/cohort under the target treatment plan is based on the synthetic model and a set of data in the tensor corresponding to the target treatment plan.

10. The method of any preceding claim, wherein the data relating to the plurality of patients or patient cohorts is data from a clinical trial for treating Alzheimer's disease.

11. The method of claim 10, further comprising using the predicted ADAS-Cog score of the target patient or target patient cohort under the target treatment plan in the results of the clinical trial.

12. The method of any preceding claim, wherein the method is for predicting missing data in a clinical trial due to a dropout.

13. A method for processing data for a clinical trial for treating a neurocognitive disease using the method of any preceding claim, the method comprising:
obtaining data relating to at least two patient cohorts, wherein a first patient cohort includes a first subset of patients undergoing a first treatment plan and a second subset of patients undergoing a second treatment plan, and a second patient cohort includes a first subset of patients undergoing the first treatment plan and a second subset of patients undergoing a third treatment plan, wherein the data includes information on the longitudinal trajectories of the patient cohorts' ADAS-Cog score over time;
encoding the data into a tensor across patient cohorts, time and treatment plan;
generating a first synthetic model of the first patient cohort and a second synthetic model of the second patient cohort using a machine learning process and the tensor; and
predicting an ADAS-Cog score of the first patient cohort under the third treatment plan using the first synthetic model, and an ADAS-Cog score of the second patient cohort under the second treatment plan using the second synthetic model.

14. The method of claim 13, wherein:
the first and second synthetic models are generated using the machine learning process based, at least in part, on a set of observed data in the tensor corresponding to the first treatment plan.

15. The method of claim 13 or claim 14, wherein:
predicting the ADAS-Cog score of the first patient cohort under the third treatment plan is based on the first synthetic model and a set of data in the tensor corresponding to the third treatment plan; and
predicting the ADAS-Cog score of the second patient cohort under the second treatment plan is based on the second synthetic model and a set of data in the tensor corresponding to the second treatment plan.

16. The method of any preceding claim, wherein the method is computer-implemented.

17. A system for predicting a patient or patient cohort's cognition score on the Alzheimer's Disease Assessment Scale-cognitive subscale (ADAS-Cog), the system comprising:
data acquisition means, configured to obtain data relating to a plurality of patients or patient cohorts, the data including information relating to the longitudinal trajectories of the plurality of patients', or patient cohorts', ADAS-Cog score over time, each patient or patient cohort having undergone a treatment plan selected from a plurality of treatment plans;
tensor generating means, configured to encode the data into a tensor across patients or patient cohorts, time and treatment plan;
synthetic model generating means, configured to generate a synthetic model of a patient or patient cohort using a machine learning process and the tensor; and
predicting means configured to predict an ADAS-Cog score of the patient or patient cohort under a target treatment plan of the plurality of treatment plans using the synthetic model.

18. The system of claim 17, wherein the system is processing data for a clinical trial for treating Alzheimer's disease, wherein:
the data acquisition means are configured to collect data from at least two patient cohorts, wherein a first patient cohort includes a first subset of patients undergoing a first treatment plan and a second subset of patients undergoing a second treatment plan, and a second patient cohort includes a first subset of patients undergoing the first treatment plan and a second subset of patients undergoing a third treatment plan, wherein the data includes information on the longitudinal trajectories of the patient cohorts' ADAS-Cog score over time;
the tensor generating means are configured to encode the data into a tensor across patient cohorts, time and treatment plan;
the synthetic model generating means are configured to generate a first synthetic model of the first patient cohort and a second synthetic model of the second patient cohort using a machine learning process and the tensor; and
the predicting means are configured to predict an ADAS-Cog score of the first patient cohort under the third treatment plan using the first synthetic model, and an ADAS-Cog score of the second patient cohort under the second treatment plan using the second synthetic model.

19. A non-transitory computer readable storage medium containing machine executable instructions which, when executed on a processor, cause the processor to perform the method of any of claims 1-16.
